(19) United States
(12) Patent Application Publication
(10) Pub. No.: US 2026/0257209 A1
(43) Pub. Date: Sep. 3, 2026

(54) METHOD FOR TREATING A CATALYST COMPRISING A ZEOLITE

(71) Applicant: IFP ENERGIES NOUVELLES, RUEIL-MALMAISON (FR)

(72) Inventors: Souad RAFIK-CLEMENT, RUEIL MALMAISON CEDEX (FR); Sylvie LACOMBE, RUEIL MALMAISON CEDEX (FR); Vincent COUPARD, RUEIL MALMAISON CEDEX (FR); Heloise DREUX, RUEIL MALMAISON CEDEX (FR)

(73) Assignee: IFP ENERGIES NOUVELLES, RUEIL-MALMAISON (FR)

(21) Appl. No.: 18/879,522

(22) PCT Filed: Jun. 19, 2023
(86) PCT No.: PCT/EP2023/066445

§ 371 Date: Dec. 27, 2024

(30) Foreign Application Priority Data
Jun. 29, 2022 (FR) FR2206555

(51) Int. Cl. B01J 29/40; B01J 35/36; B01J 35/61; B01J 35/63; B01J 37/00; B01J 37/08; C07C 2/12

(52) U.S. Cl. CPC B01J 29/40; B01J 35/36; B01J 35/615; B01J 35/633; B01J 37/0009; B01J 37/08; C07C 2/12; B01J 2229/36

(57) ABSTRACT

The present invention relates to a process for treating a catalyst comprising at least one aluminosilicate zeolite of the ZSM-5 family and a binder comprising silicon oxide, such that said treatment comprises a steam treatment of the catalyst, said steam treatment being carried out:
on the catalyst in the form of a catalytic bed of catalyst particles,
said bed being placed in a catalytic hydrocarbon conversion reactor,
with a steam-treatment gas stream passing through the said bed and comprising water vapor,
at a temperature of at least 150° C.,
and a pressure of at most 3×10⁶ Pa.

### TECHNICAL FIELD

The present invention relates to a novel process for treating material comprising a ZSM-5 zeolite. This material is advantageously used as a catalyst, as a catalyst support, but also as an adsorbent or separating agent. The invention is more particularly concerned with a use of this material as a catalyst.

### PRIOR ART

Crystalline microporous materials, such as zeolites, are solids widely used in the petroleum industry as catalysts, notably for heterogeneous catalysis of isomerization, cracking or alkylation of hydrocarbons, or for the conversion of olefins, from ethylene to propylene for example. But this type of material can also be used as a catalyst support: by adding an active element, such as copper for example, an oxidation catalyst is obtained, notably for the oxidation of ethanol to acetaldehyde, it being possible for the copper to be combined with other elements such as chromium. This type of material can also make it possible to convert alcohols into gasoline.

For catalytic oligomerization-cracking reactions at high temperature in the absence of hydrogen, ZSM-5 zeolite (of MFI framework type) is one of the most studied active phases because it has multiple advantages. The confinement of the reactants and products in the medium-pore microporous network of the ZSM-5 (apertures with 10 tetrahedral atoms) is conducive to the desired reactions while ensuring the diffusion of the products to the outside of the pores. The Si/Al ratio which dictates the number of acid sites can be adjusted by the zeolite synthesis protocol over a wide range (from 15 to 400 at./at.). This zeolite with a medium-pore aperture and a three-dimensional pore network is much less sensitive to coking than more open zeolites (for example Y and Beta zeolites of FAU and BEA framework type respectively) or one-dimensional closed zeolites (for example ZSM-22 of TON framework type). The synthesis of the zeolite also has a moderate cost which is acceptable for an industrial application. The refining and petrochemical industry is always looking for zeolite catalysts which have improved properties, notably with regard to the conversion of hydrocarbon species depending on the application (yield and/or selectivity of catalyzed reactions), and/or with regard to their stability over time, their mechanical strength, etc.

U.S. Pat. No. 4,663,492 discloses a catalyst based on zeolite ZSM-5 in the context of a process for converting methanol into gasoline, this catalyst being pretreated with steam at atmospheric pressure in order to increase its activity with respect to this conversion, this treatment being followed or preceded by a calcination.

U.S. Pat. No. 8,759,598 also discloses a zeolite-based catalyst, notably of the CHA zeolite type, in the context of a process for converting ethylene into propylene. It is recommended in this document to reduce the number of acid sites of the zeolite which are specifically located on the surface of the material. To do this, it proposes several methods, including surface silylation, steam treatment or treatment with a dicarboxylic acid.

Patent EP-3 428 249 furthermore discloses a process for converting ethylene from a cut resulting from the fractionation of an effluent from a catalytic cracking unit into propylene, aromatics and other products of interest: this conversion process is carried out in a conversion unit using a zeolite-based catalyst operating at a temperature of between 500° C. and 650° C. and at a partial pressure of olefins of between 1 and 2 bar. This catalyst comprises, for example, a ZSM-5 zeolite in a silica-type matrix.

The objective of the invention is then to improve the performance of catalysts comprising a zeolite, notably of ZSM-5 type, by targeting more particularly the conversion of ethylene or ethanol into propylene, other short olefins and other products of interest, without questioning or overly complicating their method of preparation.

In the context of the present invention, the olefins produced can be used for all applications using short olefins: monomers or polymers (polyethylene, polypropylene, polyesters) for plastics after separation of each type of compound, or else, alone or as a mixture, feedstocks for processes capable of producing fuel (jet fuel, gasoline, distillate).

The invention relates in particular to a process which accompanies the fluid catalytic cracking process (commonly referred to as FCC process). At the outlet of the reaction/regeneration assembly, there is a fractionating column, which makes it possible to separate the heavy fractions, the heavy naphtha, and the light fractions: gas, LPG and light gasolines which are at the top of the column. These light overhead fractions are then sent to a section for recovering the maximum amount of LPG and gasoline, and for optionally purifying the gas before sending it to the fuel gas. This gas, referred to as “fuel gas” contains a significant amount of ethylene, which is often burned with the fuel gas. This ethylene can be converted into propylene and into other upgradable products such as short olefins and gasoline.

### SUMMARY OF THE INVENTION

A first subject of the invention is a process for treating a catalyst comprising at least one aluminosilicate zeolite of the ZSM-5 family and a binder comprising silicon oxide, such that said treatment comprises a steam treatment of the catalyst, said steam treatment being carried out:
on the catalyst in the form of a catalytic bed of catalyst particles,
said bed being placed in a catalytic hydrocarbon conversion reactor,
with a steam-treatment gas stream passing through the said bed and comprising water vapor,
at a temperature of at least 150° C., notably of at least 180° C., for example around 200° C., or at higher temperatures, up to 400° C. and above 400° C.,
and a pressure of at most 3×10⁶ Pa, notably of at most 10⁶ Pa.

In the context of the present invention, the term “silica” or “silicon oxide” also means a silicon oxide optionally in hydrated form. This can notably be the case when the steam treatment is carried out on the solid formed into particles, for example by kneading-extrusion in the case of extrudates, and just dried after forming.

The catalyst “particles” may be in various forms depending on the chosen forming method: they may be pellets, beads or extrudates, of various, and more or less regular, shapes. The term “particles” in the context of the invention generically encompasses all possible shapes conventionally known for this type of catalyst.

The term “bed” is understood in its conventional meaning in the field of catalysts (catalyst bed): it is an arrangement in layer(s) of catalyst particles, supported by conventional mechanical means (metal screens, etc.) allowing a gaseous or liquid stream to pass through its thickness. A bed of the fixed-bed type is therefore considered here, in which the particles are not in motion and are not moved around in the reaction medium defined by the reactor in question.

The temperature should be understood here as the temperature reached by the catalyst particles, generally due to contact with the steam-treatment gas stream having this temperature.

The pressure should be understood here as the pressure inside the hydrocarbon conversion reactor.

It has thus been shown in the context of this invention that, for this type of catalyst combining a ZSM-5 zeolite and a binder of silicic type, a steam treatment at high temperature and in the reactor itself for the targeted catalytic conversion of hydrocarbon compounds gave very advantageous results, even at moderate or at atmospheric pressure: the treated catalyst makes it possible to achieve higher conversion rates and/or yields of propylene and aromatic compounds in the case of the catalysis of the ethylene to propylene conversion reaction, and also greater stability of conversion and/or yields.

But, above all, the invention proposes to treat the catalyst in the form of a catalytic bed placed in a catalytic hydrocarbon conversion reactor which will use the catalyst in question. This is referred to as “in situ” steam treatment, in so far as the catalytic bed that is being steam treated is already in the reactor in its functional position for carrying out the catalysis by coming into contact with a hydrocarbon feedstock stream and/or a stream intended to react with a feedstock stream, such as hydrogen for example: the bed is thus first passed through by the steam-treatment gas stream, then by the hydrocarbon feedstock/reactant stream. This is a very advantageous way of carrying out the steam treatment, since it limits handling and intermediate storage of the catalyst, avoids the use of a dedicated steam treatment device, and because the hydrocarbon conversion reactor is generally already equipped with all the appropriate means for implementing the invention (injection means and means for discharging gas, means for heating the chamber/gas streams, and pressurization means, means for regulating these operating conditions, notably with the aid of sensors equipping the reactor, etc.).

According to a first embodiment, the pressure at which the steam treatment is carried out is atmospheric pressure.

According to a second embodiment, the pressure at which the steam treatment is carried out is greater than atmospheric pressure, but is preferably not very high. It is preferably between 2 bar and 30 bar, i.e. between 0.2×10⁶ Pa and 3×10⁶ Pa, or between 2 and 10 bar, i.e. 0.2×10⁶ Pa and 10⁶ Pa, or else between 3 and 8 bar, i.e. between 0.3×10⁶ Pa and 0.8×10⁶ Pa.

According to one embodiment, the temperature at which the steam treatment is carried out is at least 450° C. or 500° C., notably between 500° C. and 700° C., preferably between 550° C. and 650° C., the example in the vicinity of 600° C.

According to another embodiment, the temperature at which the steam treatment is carried out is lower, notably between 150° C. and 250° C., for example between 180° C. and 200° C. In this embodiment, the pressure at which the steam treatment is carried out is advantageously greater than or equal to 10 bar, i.e. greater than or equal to 10⁶ Pa.

Preferably, the duration of steam treatment is at most 24 hours, notably at most 10 hours, or at most 5 hours, preferably between 1 hour and 3 hours. It is therefore a time that can be quite short, and therefore not too expensive in terms of catalyst preparation time or in terms of immobilization of the catalytic conversion reactor.

Preferably, the flow rate of the steam-treatment gas stream at the inlet of the reactor is between 0.01 and 0.1 NL per hour and per gram of catalyst, notably between 0.01 and 0.05 NL per hour and per gram of catalyst.

Advantageously, the steam-treatment gas stream may contain a mixture of gases comprising water vapor and at least one or more other gases chosen from N₂, CO₂, Ar, He, CH₄, air or any mixture thereof, preferably air or nitrogen.

Thus, the volume proportion of water vapor in the steam-treatment gas may be between 10% and 100%, notably between 40% and 90%, preferably between 50% and 80%.

The content of water vapor in the steam-treatment gas may be constant or changing during at least one part of the steam treatment. Choosing a constant water content is the simplest solution. Changing it, notably increasing it gradually or in stages, may also prove advantageous.

Similarly, the pressure and the temperature during the steam treatment may be constant or vary, with progressive increases, or increases in one or more stages, of the temperature or pressure in particular.

According to a first variant, the binder comprises silicon oxide, preferably consists of silicon oxide. It may be introduced during the preparation of the catalyst at least partly in the form of colloidal silica and/or in the form of precipitated silica and/or silica gel, preferably both in the form of colloidal silica and in the form of precipitated silica, or else both in the form of colloidal silica and in the form of silica gel. Such a binder is particularly inert toward the conversion reactions to be catalyzed, more than alumina for example, which is advantageous because it will increase the durability of the catalyst, without interfering with the reactions to be catalyzed: this thus avoids any disturbance and any risk of promoting a reaction that leads to unwanted by-products.

Preferably, the catalyst comprises between 20% and 80% by weight of zeolite, notably between 30% and 70% by weight of zeolite, or else between 50% and 70% by weight of zeolite, and between 20% and 80% by weight of binder, for example between 30% and 70% by weight of binder, or between 30% and 50% by weight of binder.

For mixing and shaping the catalyst from zeolite (for example in powder form) and the binder or the precursor(s) thereof (for example in powder form or in liquid form), at least one additive may be added, the function of which may notably be to help control the viscosity of the mixture before shaping (for example a viscosifying additive), very particularly when this shaping is an extrusion of the mixture in pasty form. In the final catalyst, notably when it has been heated/cured/calcined, the additive disappears, notably when it is made of organic material. It may be, for example, a cellulose derivative, notably methylcellulose.

The Si/Al atomic ratio of the zeolite contained in the catalyst is preferably between 12 and 200, notably between 35 and 180, preferably between 35 and 150.

The catalyst may also comprise at least one doping element, for example belonging to the group consisting of sodium, potassium, magnesium, calcium, phosphorus, copper, silver, manganese and molybdenum. Preferably, the doping element is phosphorus, optionally combined with one or more other elements. The content of doping element is preferably such that the atomic ratio of the element to the aluminum contained in the zeolite is less than or equal to 0.8. The doping element may be introduced by any type of preparation known to those skilled in the art, for example by dry impregnation, by excess impregnation, by chemical vapor deposition or any other type. Concerning the phosphorus element, it can for example be introduced by using one or more precursors of phosphoric acid or ammonium dihydrogen phosphate or hypophosphorous acid type.

According to a variant, the catalyst according to the invention comprises at least two ZSM-5 zeolites (which have been mixed, for example, in a preliminary mixing step for preparing the catalyst), among which at least two have different Si/Al atomic ratios, for example in a weight ratio of 10/90 to 90/10, notably 20-80 to 80-20, for example between 40-60 and 60-40. Indeed, it appears that the catalytic properties are linked at least in part to the content of Al sites in the zeolite: combining two zeolites with different Si/Al ratios can make it possible to adjust the overall acidity of the material and thus to more easily improve the catalytic properties of the catalyst and the compromise between activity and selectivity.

The shaping of the catalyst into particles can be carried out by any known method, such as, for example, kneading/extrusion, oil-drop shaping, granulation, compacting or spray drying.

The steam treatment of the catalyst may be preceded or followed by in-bed calcination of the catalyst, in the catalytic conversion reactor. The fact that calcination remains optional is very advantageous: it is therefore possible, surprisingly, to dispense with the calcination of the catalyst. A calcination step is thus replaced by a steam treatment step in the catalyst preparation process, which thus avoids extending the catalyst preparation time and complicating this preparation process.

The optional calcination of the catalyst can be carried out on the catalyst before or after the steam treatment, in the conversion reactor where the steam treatment is carried out.

The calcining conditions are for example as follows:
2° C./min increase in air at 1 Nl/h/g up to 250° C., 1 h hold;
2° C./min increase in air at 1 Nl/h/g up to 550° C., 2 h hold;
return to temperature in air.

The steam treatment of the catalyst may be preceded by (optional) drying of the catalyst particles outside of the catalytic hydrocarbon conversion reactor, notably at a temperature of at least 30° C., and preferably of at most 150° C., notably between 50° C. and 100° C., preferably between 70° C. and 90° C. Preferably, the catalyst is dried following the preparation of the catalyst (mixing of the components and then shaping). The dried catalyst can then be stored and then transferred to the plant where the conversion reactor is located. Surprisingly, it was found that the catalyst, simply dried (and not calcined for example), has very high mechanical properties, which greatly facilitate its storage, transport and installation in the conversion reactor. The in situ steam treatment then enables it to acquire the other desired properties, without requiring calcination (even if it can be provided optionally).

It has thus been found that the catalyst according to the invention, once dried, can have, before steam treatment, a mechanical strength, measured by the single pellet crush strength value, referred to as the mean SPCS, of at least 1 daN/mm, notably of at least 2 daN/mm and even of at least 2.3 to 2.5 daN/mm, in the case where the catalyst is formed into extrudates.

Another subject of the invention is a process for preparing a catalyst comprising at least one aluminosilicate zeolite of the ZSM-5 family and a binder comprising silicon oxide, said process comprising:
i) mixing at least one ZSM-5 zeolite in powder form, said binder and/or a precursor of said binder, and optionally an additive,
ii) shaping said mixture into catalyst particles,
iii) steam treating the catalyst obtained in step ii), said steam treatment being carried out:
   on the catalyst in the form of a catalytic bed of catalyst particles,
   said bed being placed in a catalytic hydrocarbon conversion reactor,
   with a steam-treatment gas stream passing through the said bed and comprising water vapor,
   at a temperature of at least 150° C.,
   and a pressure of at most 3×10⁶ Pa.

Step iii) of this preparation process may comprise a calcination of the catalyst, before or after the in-bed steam treatment in the catalytic conversion reactor, advantageously in situ therefore, like the steam treatment iii).

Step iii) of this preparation process may be preceded by a drying of the catalyst particles obtained in step ii) outside of the catalytic hydrocarbon conversion reactor, notably at a temperature of at least 30° C., and preferably of at most 150° C., notably between 50° C. and 100° C., preferably between 70° C. and 90° C.

The catalyst obtained in step ii) of this preparation process, and then dried, may have, before the steam treatment step iii), a mechanical strength corresponding to the mean single pellet crush strength value, referred to as mean SPCS, of at least 1 daN/mm, notably of at least 2 daN/mm, when the catalyst is in the form of extrudates.

Another subject of the invention is a device for carrying out the process described above, and which comprises a catalytic hydrocarbon conversion reactor, placed in which is a catalytic bed of catalyst particles comprising at least one aluminosilicate zeolite of the ZSM-5 family, a binder comprising silicon oxide, and such that the reactor comprises means for injecting and discharging steam-treatment gas comprising water vapor. As mentioned above, this is an “in situ” implementation of the invention, where the steam-treatment gas flow will pass through the catalytic bed, as will the hydrocarbon feedstock and/or a stream of reactant to be converted afterwards.

Another subject of the invention is the catalyst obtained by the treatment process or the treatment device described above.

Another subject of the invention is a process for converting ethylene or ethanol into propylene, other short olefins and gasoline or aromatic compounds, which uses a catalyst as treated and described above. The feedstock to be treated is brought into contact with the catalyst under the conditions of the conversion process, after optional activation of the catalyst.

The optional activation of the catalyst in the form of a bed of particles can take the form of a drying-type heat treatment at high temperature in air, and/or a calcination aimed at burning any traces of oil or grease that may be present.

The operating conditions of the ethylene conversion process are described, for example, in patent EP-3 428 249, to which reference should be made for more details and which can be summarized as follows: use is made of a unit for the conversion of ethylene to propylene and other products of interest, which is a catalytic unit using a zeolite-based catalyst operating at a temperature of between 450° C. and 650° C. and under an olefin partial pressure of between 1 and 4 bar, i.e. between 0.1×10⁶ Pa and 0.4×10⁶ Pa, with an hourly weight velocity (weight of olefinic feedstock per weight of catalyst and per hour) of between 0.1 and 10 h⁻¹, preferably between 1 and 7 h⁻¹.

In the case where the reaction feedstock is ethanol, the latter is initially dehydrated to ethylene, which is converted, under the same operating conditions as those of the dehydration and over the same catalyst, in the presence of water released by the dehydration reaction, to oligomers, i.e. to C3-C6 light olefins. The operating conditions of the ethanol conversion process are described, for example, in patent FR-2 948 937, to which reference may be made for further details and which can be summarized as follows: for example, a temperature of between 300° C. and 600° C., preferably between 450° C. and 575° C., under a pressure of between 0.1×10⁶ and 1.5×10⁶ Pa, preferably between 0.1×10⁶ and 0.5×10⁶ Pa, with an hourly weight velocity (weight of ethanol feedstock per weight of catalyst and per hour) of between 0.1 and 10 h⁻¹, preferably between 1 and 4 h⁻¹.

### DESCRIPTION OF THE EMBODIMENTS

The invention relates to a catalyst comprising at least one zeolite or aluminosilicate of the ZSM-5 family and a binder comprising silicon oxide.

It is implemented in the following examples using three different catalysts having the following formulations:

### Catalyst A

It is in the form of extrudates (cylinders with a diameter of 1.4 mm, and a length of between 2 and 6 mm) containing 60% by weight of ZSM-5 zeolite with an Si/Al atomic ratio equal to 140 (commercially available from Zeolyst, under the commercial reference CBV28014) and 40% of silicon oxide binder obtained from two sources of silica:
on the one hand from 20% (expressed on a dry basis) of silica gel available under the trade name Siliaflash C60 (particle size <20 μm) and sold by SiliCycle;
on the other hand from 20% (expressed on a dry basis) of colloidal silica, available under the trade name LUDOX™ AS-40 sold by Grace, and which is a 40 wt % suspension of colloidal silica in water.

### Catalyst Preparation

The preparation of the catalyst from the zeolite and the two sources of silica was carried out with a forming additive, here a cellulose derivative: METHOCEL™, in a proportion of 4% by weight relative to all of the dry solid, available from DuPont, and which is a water-soluble polymer derived from cellulose.

The zeolite, silica sources, additive and water were mixed and kneaded. When the paste had the appropriate rheology, it was extruded through a die.

### Catalyst Drying

After shaping in the form of extrudates, and before the steam treatment according to the invention, the catalyst was dried at 80° C. in air in a drying oven for 24 hours. As a variant, the drying may be shorter (only a few hours, for example 5 to 10 hours) or longer, and may be carried out at slightly higher temperatures (90-100° C.) or lower temperatures (60-70° C.).

It should be noted that here the extrudate is cylindrical in shape, but alternatively it can have another shape, for example be trilobate or quadrilobate in shape.

### Steam Treatment of the Catalyst

The steam treatment according to the invention is carried out on catalyst A in the form of particles placed in a catalytic bed in a catalytic hydrocarbon conversion reactor, said catalytic bed being passed through by a steam-treatment gas stream comprising water vapor, at a high temperature according to one embodiment, namely at least 400° C. and a pressure of at most 3×10⁶ Pa, notably 10⁶ Pa, for a time of at most 10 hours, preferably at most 4 hours.

Alternatively, as indicated above, the steam treatment according to the invention can also be carried out at a lower temperature (from 150° C. or from 180° C.) and/or at a higher pressure, notably up to 30 bar.

The experiments were conducted in a laboratory reactor simulating the chamber of a catalytic conversion reactor: to do this, 10 g of catalyst are loaded into a tubular steel reactor so that a steam treatment fluid can flow through it, in this case a gas stream containing water vapor and possibly other gases of air or nitrogen type, and so as to withstand high pressures. The uncompacted thickness of the catalytic bed is 20 cm. This reactor is placed in a heating chamber. The water is vaporized upstream of the reactor in another tubular reactor (called a “vaporizer”) filled with silicon carbide, and the vaporizer-steam treatment reactor connecting lines are heated to 220° C. The gas injected at the same time as the water vapor also passes through the vaporizer.

The steam treatment according to the invention is defined by a temperature T in ° C. of the catalytic bed, a pressure P in bar/Pa (in the tubular reactor containing the catalyst), a treatment time D in hours, which corresponds to the time during which the temperature T has been reached and maintained (duration of the temperature hold therefore), a flow rate Q of steam-treatment gas passing through the catalytic bed expressed in NL/h/g (normal liters per hour and per gram of catalyst), and a volume percentage of water vapor in the steam-treatment gas comprising a mixture of water vapor and air.

The steam treatment conditions for catalyst A are as follows:
5° C./min increase in air at 1 Nl/h/g up to 150° C., 1 h hold, then increase to 600° C.;
passage in 50/50 vol % H₂O/air, 4 h hold;
return to temperature in air;
pressure: atmospheric P

### Optional Calcination of the Catalyst

It can be carried out on the catalyst before or after the steam treatment, in the conversion reactor where the steam treatment is carried out.

The calcining conditions are as follows:
−2° C./min increase in air at 1 Nl/h/g up to 250° C., 1 h hold;
−2° C./min increase in air at 1 Nl/h/g up to 550° C., 2 h hold;
return to temperature in air.

### Catalyst B

Catalyst B is in the form of extrudates (cylinders with a diameter of 1.4 mm, and a length of between 2 and 6 mm) containing 60% by weight of ZSM-5 zeolite with an Si/Al atomic ratio equal to 140 (commercially available from Zeolyst, under the commercial reference CBV28014) and 40% of silicon oxide binder obtained from two sources of silica:
on the one hand from 20% (expressed on a dry basis) of silica gel available under the trade name Siliaflash C60 (particle size 40-63 μm) and sold by SiliCycle;
on the other hand from 20% (expressed on a dry basis) of colloidal silica, available under the trade name LUDOX™ AS-40 sold by Grace, and which is a 40 wt % suspension of colloidal silica in water.

The catalyst is prepared according to the same protocol as that applied for catalyst A.

### Catalyst C

Catalyst C is in the form of extrudates (cylinders with a diameter of 1.4 mm, and a length of between 2 and 6 mm) containing 60% by weight of ZSM-5 zeolite with an Si/Al atomic ratio equal to 140 (commercially available from Zeolyst, under the commercial reference CBV28014) and 40% of silicon oxide binder obtained from two sources of silica:
on the one hand from 20% (expressed on a dry basis) of precipitated silica powder, available under the trade name NYASIL™ 20 sold by Nyacol Nano Technology) and which is a nanoscale structured amorphous silica powder;
on the other hand from 20% of colloidal silica, available under the trade name LUDOX™ AS-40 sold by Grace, and which is a 40 wt % suspension of colloidal silica in water.

The catalyst is prepared according to the same protocol as that applied for catalyst A.

### Example 1 (According to the Invention)

Catalyst A was prepared, dried and then steam treated as indicated above.

### Example 2 (According to the Invention)

Catalyst B was prepared, dried and then steam treated as indicated above.

### Example 3 (According to the Invention)

Catalyst C was prepared, dried and then steam treated as indicated above.

### Example 4 (Comparative)

Catalyst A was prepared and then dried under the same conditions as in example 1, but was not steam treated. However, it was calcined in the conversion reactor.

### Example 5 (Comparative)

Catalyst B was prepared and then dried under the same conditions as in example 2, but was not steam treated. However, it was calcined in the conversion reactor.

### Example 6 (Comparative)

Catalyst C was prepared and then dried under the same conditions as in example 3, but was not steam treated. However, it was calcined in the conversion reactor.

Table 1 below gives textural characteristics of the catalysts treated according to examples 1 to 6:
Vp Hg inf 7 μm: corresponds to the pore volume with a diameter of less than 7 micrometers measured by mercury porosimetry;
V macro Hg: corresponds to the volume representative of the macropore volume (pore diameter between 50 nm and 7 μm) measured by mercury intrusion;
V meso Hg: corresponds to the volume representative of the mesopore volume (pore diameter between 2 and 50 nm) measured by mercury intrusion;
S BET: corresponds to the specific surface area measured by nitrogen adsorption isotherm;
Vμ N₂: corresponds to the micropore volume obtained (pore diameter less than 2 nm) calculated from the nitrogen adsorption isotherm by the t-plot method;
mean PPC: corresponds to the mean breaking force in daN per mm length. The nitrogen adsorption isotherm was measured at 77 K according to ASTM D3663-03, using a Micromeritics 2020 ASAP apparatus. Just before the analysis, the sample is placed under high vacuum (1×10⁻⁵ mbar) for 1 hour at 100° C. and then for 4 hours at 450° C.

The pore volume is measured by mercury porosimetry according to the standard ASTM D4284-03. Just before the analysis the sample is placed n an oven at 250° C. overnight.

The SPCS value is obtained via a standardized test (standard ASTM D4179-01) that consists in subjecting a material in the form of a millimetric scale object to a compressive force causing it to break. This test is thus a measurement of the tensile strength of the material. The analysis is repeated on a certain number of solids taken individually and typically on a number of solids of between 10 and 200. The mean of the lateral breaking forces measured is the mean SPCS.

TABLE 1

| | | | | | | | SPC |
|---|---|---|---|---|---|---|---|
| | | | | | | SPCS | of the |
| | Vp Inf | Vmacro | Vmeso | | | after | final |
| | 7 μm | Hg | Hg | S BET | Vμ N₂ | drying | catalyst |
| Example | (cc/g) | (cc/g) | (cc/g) | (m²/g) | (cc/g) | (daN/mm) | (daN/mm) |
| Example 1 | 0.38 | 0.15 | 0.23 | 331 | 0.099 | 2.4 | 0.36 |
| Example 2 | 0.33 | 0.12 | 0.21 | 332 | 0.097 | 2.7 | 0.32 |
| Example 3 | 0.30 | 0.13 | 0.17 | 288 | 0.108 | 2.9 | 0.32 |
| Example 4 | 0.41 | 0.17 | 0.24 | 324 | 0.107 | 2.4 | 0.10 |
| (comp.) | | | | | | | |
| Example 5 | 0.39 | 0.15 | 0.22 | 321 | 0.110 | 2.5 | 0.11 |
| (comp.) | | | | | | | |
| Example 6 | 0.23 | 0.09 | 0.13 | 291 | 0.104 | 2.9 | 0.31 |
| (comp.) | | | | | | | |

It is found that the catalyst of example 1 (dried and then steam-treated) has porosity characteristics similar to the catalyst of example 4 (dried and then calcined): the steam-treatment slightly modifies the textural properties of the catalyst.

However, it can be seen that the catalyst dried only according to example 1 has an SPCS value more than 6 times higher than that of the catalyst of example 1 which was subsequently steam-treated in situ: it is confirmed that a heat treatment of the steam treatment type modifies the mechanical strength of the catalyst, and that it is very advantageous to handle, transport and install the catalyst in the catalytic conversion reactor before the steam treatment thereof. Once in place in the reactor, it is indeed no longer necessary for the catalyst to maintain a very high level of strength, and the steam treatment can then be carried out, which is itself beneficial to the catalytic performance of the catalyst. The same trend is observed on catalysts 2 and 5, and also on catalysts 3 and 6.

Measurements were also made to quantify the catalytic performance of the examples for converting ethylene predominantly to propylene.

After the in situ heat treatment (steam treatment or calcination depending on the examples), the reactor is then inerted under a nitrogen stream with a flow rate of 6 NL/h and then the hydrocarbon feedstock consisting of pure ethylene is injected.

For each test, 7 g of ethylene feedstock are injected per hour and per gram of catalyst. The reaction conditions used are a temperature of 500° C. and a pressure of 0.17 MPa. At the reactor outlet, the entire effluent maintained in gaseous form by heating the transfer lines is analyzed by gas chromatography.

The catalytic performance properties thus obtained for each of the catalysts are given in table 2 below. They are expressed using the following criteria:
the conversion of ethylene X(ethyl.) and the yield of propylene Y(propyl.) expressed as follows:

$$X ( ethyl . ) = 1 - ( weight fraction of ethylene in the effluent )$$

$$Y ( propyl . ) = weight fraction of propylene in the effluent$$

the purity of the propylene in the cut consisting of propane and propylene P(propyl.), corresponding to 55% conversion of ethylene, expressed as follows:

$$P ( propyl . ) = weight fraction of propylene in the effluent / weight fractions of propylene and propane in the effluent$$

The activity of the catalyst is characterized by the initial ethylene conversion X. The selectivity of the catalyst is characterized by the propylene purity P and the propylene yield Y.

The stability of catalyst performance is quantified as follows:

$$conversion stability = ( initial conversion - conversion after 15 h under feedstock ) / initial conversion$$

$$Stability of the propylene yield = ( initial propylene yield - propylene yield after 15 h under feedstock ) / initial propylene yield$$

The catalytic performance values thus obtained for each of the catalysts are given in table 2 below:

TABLE 2

| | | | | | Y | Loss of | P |
|---|---|---|---|---|---|---|---|
| | | X | Loss of | | (propyl.) | propylene | (propyl.) |
| | | (ethyl.) | conversion | | after 15 | yield after | for |
| | X | after 15 h | after 15 h | Y | hours | 15 h | X(ethyl) |
| Example | (ethyl.) | under | (relative | (propyl.) | under | (relative | conversion |
| No. | initial | feedstock | %) | initial | feedstock | %) | of 55% |
| 1 | 63 | 55 | 13% | 21 | 19 | 10% | 94 |
| 2 | 61 | 47 | 23% | 19 | 16 | 16% | 95 |
| 3 | 60 | 40 | 33% | 18 | 13 | 28% | 95 |
| 4 | 60 | 5 | 92% | 17 | 1.5 | 91% | 93 |
| (comp.) | | | | | | | |
| 5 | 58 | 4 | 93% | 19 | 1.2 | 94% | 92 |
| (comp.) | | | | | | | |
| 6 | 59 | 10 | 83% | 17 | 5 | 71% | 95 |
| (comp.) | | | | | | | |

It can be seen that the catalyst treated according to the invention (example 1) compared to the non-steam-treated reference catalyst (example 4) has:
a similar initial conversion but a significant gain in conversion stability;
a similar initial propylene yield, but a significant gain in the stability of this yield;
a propylene purity at least equivalent to the reference.

1. A process for treating a catalyst comprising at least one aluminosilicate zeolite of the ZSM-5 family and a binder comprising silicon oxide, the process comprising:
a steam treatment of the catalyst, wherein said steam treatment is carried out:
on the catalyst in the form of a catalytic bed of catalyst particles,
said bed being placed in a catalytic hydrocarbon conversion reactor,
with a steam-treatment gas stream passing through the bed comprising water vapor,
at a temperature of at least 150° C., and
at a pressure of at most 3×10⁶ Pa.

2. The process as claimed in claim 1, wherein the pressure at which the steam treatment is carried out is atmospheric pressure.

3. The process as claimed in claim 1, wherein the pressure at which the steam treatment is carried out is between 0.2×10⁶ Pa and 3×10⁶ Pa or between 0.2×10 6 Pa and 10⁶ Pa.

4. The process as claimed in claim 1, wherein the temperature at which the steam treatment is carried out is at least 450° C. or 500° C., notably between 500° C. and 700° C., preferably between 550° C. and 650° C., or between 150° C. and 250° C., notably between 180° C. and 200° C.

5. The process as claimed in claim 1, wherein the duration of the steam treatment is at most 24 hours, notably at most 10 hours, or at most 5 hours, preferably between 1 hour and 3 hours.

6. The process as claimed in claim 1, wherein the flow rate of the steam-treatment gas stream is between 0.01 and 0.1 NL per hour and per gram of catalyst, notably between 0.01 and 0.05 NL per hour and per gram of catalyst.

7. The process as claimed in claim 1, wherein the steam-treatment gas stream contains a gas mixture comprising water vapor and at least one other gas, notably air and/or nitrogen, the content of water vapor in the steam-treatment gas being constant or changing during at least one part of the steam treatment.

8. The process as claimed in claim 7, wherein the volume proportion of water vapor in the steam-treatment gas is between 10% and 100%, notably between 40% and 90%, preferably between 50% and 85%.

9. The process as claimed in claim 1, wherein the binder comprises silicon oxide introduced into the catalyst at least partly in the form of colloidal silica and/or in the form of precipitated silica, preferably both in the form of colloidal silica and in the form of precipitated silica.

10. The process as claimed in claim 1, wherein the catalyst comprises between 20% and 80% by weight of zeolite, notably between 30% and 70% by weight of zeolite, and between 20% and 80% by weight of binder, notably between 30% and 70% by weight of binder.

11. The process as claimed in claim 1, wherein the Si/Al atomic ratio of the ZSM-5 zeolite is between 12 and 200, notably between 35 and 180, preferably between 35 and 150.

12. The process as claimed in claim 1, wherein the catalyst comprises at least one doping element, notably belonging to the group consisting of sodium, potassium, magnesium, calcium, phosphorus, copper, silver, manganese and molybdenum.

13. The process as claimed in claim 1, wherein the catalyst comprises at least two ZSM-5 zeolites, among which at least two have different Si/Al atomic ratios.

14. The process as claimed in claim, wherein the steam treatment of the catalyst is preceded or followed by a calcination of the catalyst as a bed in the catalytic conversion reactor.

15. The process as claimed in claim 1, wherein the steam treatment of the catalyst is preceded by a drying of the catalyst particles outside of the catalytic hydrocarbon conversion reactor, notably at a temperature of at least 30° C., and preferably of at most 150° C., notably between 50° C. and 100° C., preferably between 70° C. and 90° C.

16. The process as claimed in claim 15, wherein the dried catalyst has, before steam treatment, a mechanical strength corresponding to the mean single pellet crush strength value, referred to as mean SPCS, of at least 1 daN/mm, notably of at least 2 daN/mm, in the case where the catalyst is formed into extrudates.

17. A process for preparing a catalyst comprising at least one aluminosilicate zeolite of the ZSM-5 family and a binder comprising silicon oxide, said process comprising:
i) mixing at least one ZSM-5 zeolite in powder form, said binder and/or a precursor of said binder, and optionally an additive,
ii) shaping said mixture into catalyst particles,
iii) subjecting the catalyst particles obtained in step ii) to a steam treatment, said steam treatment being carried out:
on the catalyst in the form of a catalytic bed of catalyst particles,
said bed being placed in a catalytic hydrocarbon conversion reactor,
with a steam-treatment gas stream passing through the said bed and comprising water vapor,
at a temperature of at least 150° C.,
and a pressure of at most 3×10⁶ Pa.

18. A device for carrying out the process as claimed in claim 1, wherein the device comprises a catalytic hydrocarbon conversion reactor, placed in which is a catalytic bed of catalyst particles comprising at least one aluminosilicate zeolite of the ZSM-5 family, a binder comprising silicon oxide, and in that the reactor comprises means for injecting and discharging steam-treatment gas comprising water vapor.

19. A process for the catalytic conversion of a feedstock comprising ethylene or ethanol into propylene and gasoline or aromatic compounds, comprising contacting said feedstock with the catalyst treated as claimed in claim 1.

20. The process as claimed in claim 1, wherein the binder consists of silicon oxide introduced into the catalyst at least partly in the form of colloidal silica and/or in the form of precipitated silica.
